(19) United States
(12) Patent Application Publication
(10) Pub. No.: US 2012/0059440 A1
(43) Pub. Date: Mar. 8, 2012

(54) PORTABLE LIGHT HAIR RESTORATION HELMET

(75) Inventor: Tamim Said HAMID, Pleasanton, CA (US)

(73) Assignee: THERADOME INC., Pleasanton, CA (US)

(21) Appl. No.: 12/950,009

(22) Filed: Nov. 19, 2010

Related U.S. Application Data
(60) Provisional application No. 61/264,449, filed on Nov. 25, 2009.

(51) Int. Cl. A61N 5/06

(52) U.S. Cl. 607/91

(57) ABSTRACT

The present invention provides an apparatus for providing increased hair growth and healthier scalp. The apparatus comprises of a swappable-zoned flexible helmet to provide increased hair growth, shinier hair, denser hair and healthier scalp using no medication and zero side-effects. The flexible Helmet device consists of a laser or LED array (from 25 to 300 tiny lasers or 25-300 LEDs) which bio-stimulate the scalp and encourages hair growth. The present invention provides the treatment by subjecting the affected scalp to flexible helmet equipped with the laser or LED array, 3 times a week for a minimum of 20 minutes per session to enable the absorption of laser or light from LED to the scalp for effective treatment.

### FIELD OF INVENTION

The present invention relates to an apparatus for the therapeutic light treatment of scalp. The present invention also relates to an encouragement of hair augmentation. In particular, the present invention is directed to the bio-stimulating treatment of hair and scalp by providing a flexible helmet equipped with the laser or light emitting diode (LED) array to provide increased hair growth, shinier hair, denser hair and the healthier scalp.

### BACKGROUND OF INVENTION

The loss of hair has traditionally been a problem for a large percentage of the population. The main causes for hair loss is the tightening of the scalp where nutrients and oxygen are no longer available or accessible to the follicles, therefore miniaturization of the follicles occur and eventually death to the hair follicles. The micro-circulation reduction results in ongoing atrophy which causes the loss of functionality of the hair follicle, which gradually disables the hair generation.

Early attempts to address this problem focused on the hair pieces (i.e. wigs or toupees), but they have several drawbacks. In particular, they have an unnatural appearance and always have a risk of being loosened or damaged. Methods of manually exercising the scalp has been greatly published and proven to be a great method of preserving hair and facial youthfulness. The disadvantages are that they are quite laborious, boring and time consuming to do on a regular basis. Most people do not last more than two weeks doing these manual scalp exercises even though there is enough data and testimonials that they do work. Other solutions to baldness and hair loss involves various modern drugs, which can take the form of either an ingestible medication or a tropical ointment but, these have proved out to be not effective as they produces harmful side effects in the body.

Introducing a laser or LED light that bio-stimulates the scalp, results in hair growth and stopping of shedding and thickening of hair follicles. Devices having structures similar to a large permanent fitted floor mounted or chair mounted have been developed which contains multiple laser or LED assemblies. It has been found that there are several disadvantages associated with this approach, as these are typically very large and expensive commercial devices which are permanently installed at a place and in order to advantage them, the user has to visit the place regularly where these devices are located. This causes the user an expensive and troublesome treatment. Additionally, such treatment requires trained operator.

Prior art attempts to provide hand held devices which have resulted in many undesirable drawbacks as these devices tends to be large, bulky and complex due to the use of multiple laser modules and/or fiber optics. Moreover, the alignment of multiple lasers in the device makes them tough to manufacture.

There are bulky head covering system and hand held hair devices. Laser Combs for stimulating hair growth which are proven to be more hectic and complicated in working. The Laser Combs have inconsistent laser therapy, which causes the arms tired during working and are always associated with the limited efficacy (9 lasers) and Safety Issues with them. So, there is the need of a portable, simple, easy-to-use and inexpensive laser or LED device which provides consistent laser/LED therapy that an individual can use at his or her convenience with the added advantage of being efficient during usage.

U.S. patent application Ser. Nos. 11/563,675, 11/212,916, 11/853,937 describes hair comb for achieving the hair growth.

Hoods and helmets are a logical choice in attempting to bath the scalp with a few laser light beams. The Laser Hoods present in the market now a days are inconvenient to attend 2-3 weekly sessions. Moreover, they are expensive ($3K-5K per year) and are again risked with the Safety Issues as most laser restoration devices can be harmful, if used carelessly.

Therefore, there is the need of an apparatus which could eliminate these drawbacks and provide an apparatus which is cost effective along with additional advantage of its being user friendly as it can be used any where depending on the choice of user.

### SUMMARY OF THE INVENTION

The present invention provides an apparatus for therapeutic light treatment of scalp and providing increased hair growth and healthier scalp. The apparatus comprises of a flexible helmet equipped with the array of light emitting devices, control mechanism and sensing unit to provide increased hair growth, shinier hair, denser hair and healthier scalp using no medication and zero side-effects.

An embodiment of the present invention discloses an apparatus for therapeutic light treatment of zones of a user's scalp. The apparatus comprises at least one swappable zones to cover one or more specific zone to increase the amount of energy and for simultaneously stimulating that zone of the users scalp, a plurality of Light emitting devices arranged inside the swappable zone, a sensing unit configured for sensing one of a proximity of a swappable zone and generation of heat, and a control mechanism. The control mechanism is operatively coupled to plurality of Light emitting devices and the sensing unit for controlling the operation and providing treatment to swappable zones.

Another embodiment of present invention discloses an apparatus for therapeutic light treatment of a user's scalp. The apparatus comprises a helmet designed to cover a user's frontal scalp, parietal scalp, and temporal scalp. The helmet comprises plurality of light emitting devices configured to maintain a substantially even distance from the user's scalp, and configured to expose at least the user's frontal scalp, parietal scalp, and temporal scalp to therapeutic levels of light illumination. The helmet further comprises at least one proximity sensor to prevent operation of plurality of light emitting devices when the helmet is not being worn and wherein upon placement on a user's scalp and initiation of therapeutic light treatment, at least some of the light emitting devices will operate, thus exposing at least selected regions of the user's scalp to therapeutic levels of light illumination.

Another one embodiment of present invention discloses a battery powered apparatus for therapeutic light treatment of a user's scalp for baldness or pattern baldness. The apparatus comprises a helmet designed to cover a user's frontal scalp, parietal scalp, and temporal scalp. The helmet comprises a plurality of light emitting devices configured to maintain a substantially even distance from the user's scalp, and configured to expose at least the user's frontal scalp, parietal scalp, and temporal scalp to therapeutic levels of light illumination, wherein the light emitting devices are between 25 to 300 Light Emitting Diodes, Laser Diodes, or vertical-cavity surface-emitting laser (VCSEL) devices capable of delivering between 3 to 6 joules of light energy per square centimeter of scalp during the course of a treatment session that lasts no longer than an hour. The helmet further comprises at least one proximity sensor to prevent operation of plurality of the light emitting devices when the helmet is not being worn. The helmet furthermore comprises control mechanism to allow at least illumination of the user's frontal scalp, parietal scalp, and temporal scalp to be controlled independently. The helmet furthermore comprises at least one heat sensor to detect the temperature of the user's scalp, and a regulating mechanism designed to reduce or terminate light exposure to at least those regions of the scalp where the detected temperature exceeds a preset threshold, wherein the apparatus further comprises a display system that shows time per session and total light emitting device treatment time, wherein the apparatus further comprises a user interface to allow the user to select apparatus On/Off status and which part(s) of the scalp will be treated and wherein upon placement on a user's scalp and initiation of therapeutic light treatment, at least some of the light emitting devices will operate, thus exposing at least selected regions of the user's scalp to therapeutic levels of light illumination.

The one object of the primary invention is to provide the flexible helmet consists of a laser or LED array (from 25 to 300 tiny lasers or 25-300 LEDs) which bio-stimulate the scalp and encourages hair growth. The present invention provides the treatment by subjecting the affected scalp to flexible helmet equipped with the laser or LED array 3 times a week for a minimum of 20 minutes per session to enable the absorption of laser or light to the scalp for effective treatment. Treatment can be repeated as needed.

Another object of the present invention is to provide an apparatus and a method that is sufficiently effective in terms of laser or LED light intensity and treatment duration.

### BRIEF DESCRIPTION OF DRAWINGS

For a more complete understanding of the invention, reference is now made to the following description, taken in conjugation with the accompanying drawings, in which:

FIG. 1a shows the side view of the flexible Helmet in accordance with the present invention.

FIG. 1b shows the front view of the flexible Helmet in accordance with the present invention

FIG. 2 shows the different inner parts of the flexible Helmet in accordance with the present invention.

FIG. 3 shows the connection of Light Emitting Diodes (LED), Laser Diodes, or vertical-cavity surface-emitting laser (VCSEL) devices and cables to shell and platform in the flexible Helmet in accordance with the present invention.

FIG. 4 shows the coverage of the head by the flexible Helmet in accordance with the present invention.

FIG. 5 shows Laser/LED/VCSEL diffusion Pattern by the flexible Helmet in accordance with the present invention.

### DETAILED DESCRIPTION ACCOMPANYING DRAWINGS

The embodiments of the present invention will be described in detail with reference to the accompanying drawings. However, the present invention is not limited to the embodiments. The present invention can be modified in various forms. The embodiments of the present invention are only provided to explain more clearly the present invention to the ordinarily skilled in the art of the present invention. In the accompanying drawings, like reference numerals are used to indicate like components.

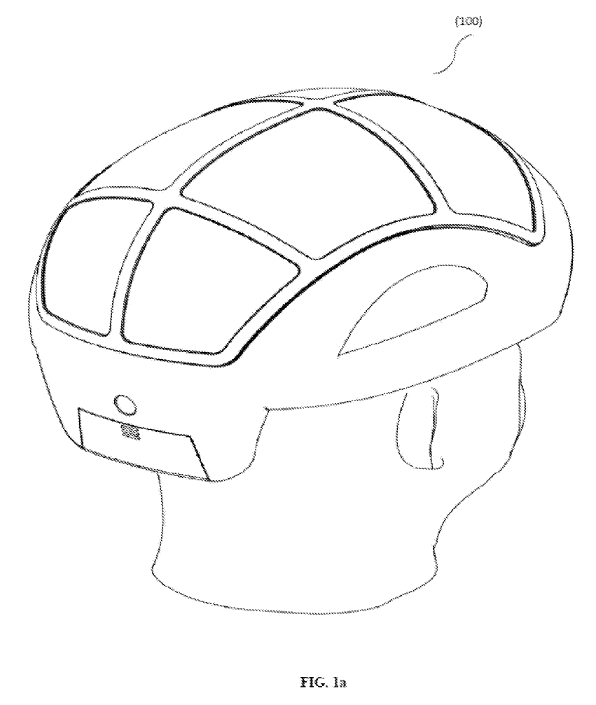

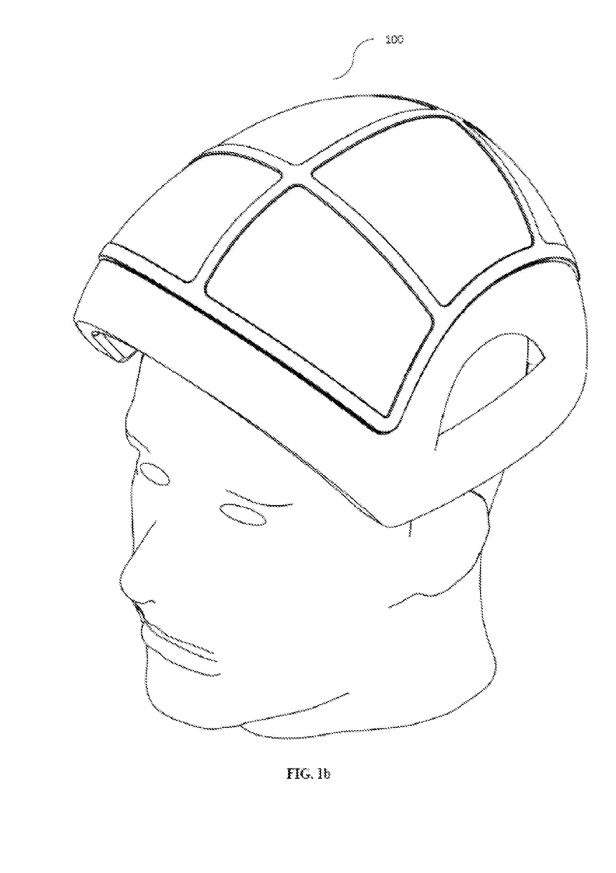

FIG. 1 illustrates a front view (FIG. 1a) and back view (FIG. 1b) of helmet according to an embodiment of the present invention. The invention provides swappable-zoned flexible helmet (100) which is able to substantially increase the amount of energy and simultaneously stimulate the scalp muscles deep within the cranial theca. These swappable zones include light emitting devices such as LED, Laser diode or vertical-cavity surface-emitting laser (VCSEL) devices for the therapeutic treatment of scalp. By creating these swappable zones, the lasers diodes or LEDs or VCSEL have more power and energy to therapeutically increase the follicle stimulating benefits of LLLT (Low Level Laser Therapy or Low Level LED Therapy) for hair loss. The added benefits of this extra stimulation create more production of Nitric oxide, S.O.D., as well as increased ATP production in each of the scalp cells.

Another embodiment of the present a flexible Helmet (100) or head covering (100) which is cost effective, efficient and safer to use which allows the user to maximize the effects of the laser or LEDs or VCSEL and the stimulation of the scalp muscles in order to generate the most amount of hair in the shortest amount of time.

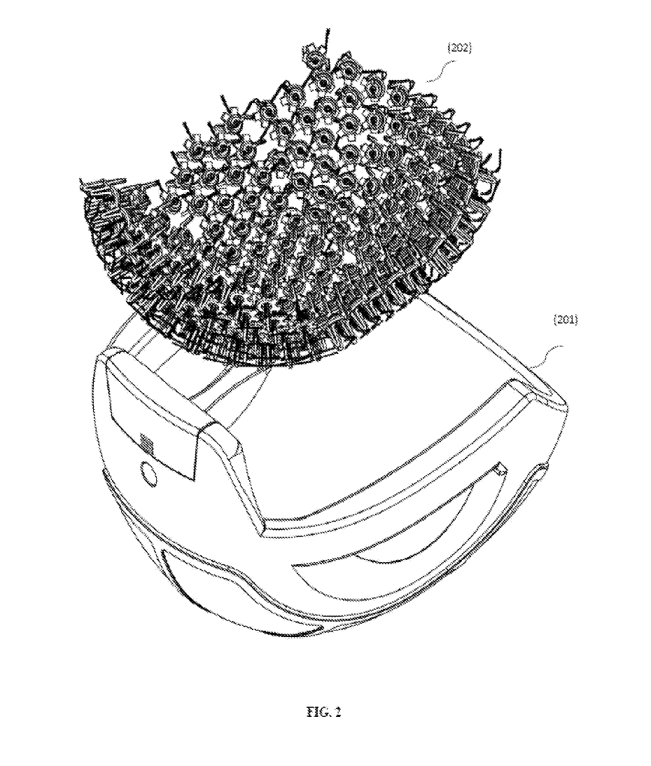

FIG. 2 illustrates an inner view helmet (201) according to another embodiment of the present invention. The flexible Helmet (201) is designed with about 25-300 tiny light emitting devices (201) such as laser diode, LED or VCSEL that project an unfocused laser light onto the scalp of a hair loss subject. The 25-300 tiny lasers, LED or VCSEL devices (201) lights are fixed into specially designed flexible helmet (201) in matching the head contour. These laser diode, LED or VCSEL lights get absorbed by the scalp and the longer a person wears the flexible Helmet, the more energy the scalp tissue absorbs. This absorbed energy is measured in joules per centimeter square. The optimum energy delivered to the scalp should be between 3-6 joules in a given session but our discovery and findings suggest that substantial improvements in hair growth is experienced when the scalp has absorbed about 4-6 joules/cm² of energy.

Laser or LED Efficacy is calculated by using the following formula:

Total Joules=Watt per diode×# of Diodes×Time (Secs)

Energy Density (J/cm²)=Watt per diode×# of Diodes×Time (Secs)/Coverage area (cm²)

The flexible Helmet (100) stimulates the scalp muscles using specific zones of laser diodes, LED or VCSEL devices (202). It is provided with the three unique treatment zones for maximum treatment and more effective method to treat bald areas. To be more specific, when all of the 25 or so lasers/LEDs/VCSEL (202) are turned on and bio-stimulating the entire scalp, most users can tolerate a long therapy session of typically up to 45 minutes, but when the laser diodes or LEDs or VCSEL (202) are separated into three unique zones namely, frontal scalp, parietal scalp and temporal scalp (300/3), and only one zone is turned on at a time for up to 20 minutes at a time, the user experiences an intense stimulation of their scalp muscles to the point where it is almost intolerable beyond 20 minutes each given zone which results in the bringing of more blood to the whole cranial area (skin on the scalp where the follicles resides). It strengthens the walls of the capillaries that nourish the hair follicles, and it increases the flexibility and elasticity of the whole scalp treating the Males and Females Pattern Baldness.

Treatment Time is calculated by the following formula:

Treatment Time (Secs)=Energy Density (J/cm²)/Output Power Density (W/cm²)

The flexible Helmet (201) is a lightweight apparatus weighing less than 3 lbs making it easy and comfortable for usage. In a further embodiment of the present invention, the flexible Helmet (201) has a display system which shows Timer per session and Total Laser or LED or VCSEL devices Treatment Time. It is powered with the battery with AC charger (110V/220V). The apparatus is Wire Free having minimal wiring with the wireless remote control system. The flexible Helmet (201) have Multiple Zones (three zones, namely Frontal scalp, Parietal scalp and Temporal scalp) which will allow the user to select which part of the scalp will be treated, allowing more power and treatment time for those areas which are destined to and simultaneously eliminating the undesired exposure of the other areas.

In another embodiment of the present invention, the flexible Helmet device has control mechanism to On/Off a specific zone and sensing unit for sensing the user scalp. The sensing unit in the flexible Helmet has plurality of sensors such as Proximity sensor and Heat sensor. The Proximity sensor causes the lasers or LEDs or VCSEL (202) devices to turn on when it senses the scalp otherwise will remain off until user wears the flexible Helmet while the Heat sensor of the flexible Helmet regulates the temperature on scalp. The distance from laser or LED or VCSEL to scalp will determine the coverage and effective hot spots. In a further embodiment of the present invention, the uniform distance between scalp and flexible Helmet (201) is maintained by using 1 cm spacer. The apparatus is equipped with an adjustable timer and alarm for session. The flexible Helmet (100) has shown to provide increased hair growth, shinier hair, denser hair and healthier scalp using no medication and zero side-effects.

In a preferred embodiment, the flexible Helmet (100) weighing 2 lbs attached with 25-300 tiny laser diodes or LEDs or VCSELs is used to fit in the head size to cover the top of the head or the crown area (up to Norwood 5A) projecting an unfocused laser or LED light onto the scalp which bio-stimulate the scalp and encourages hair growth. In another embodiment of the present invention, a matrix of spacer that keeps the laser or LED lights from a human scalp for a specified distance. The flexible Helmet is attached with the AC charger of 110V. The Proximity sensor causes the lasers or LEDs to turn on when it senses the scalp while the heat sensor regulates temperature on scalp. The alarm rings up after the completion of adjusted time for the session. The flexible Helmet (100) user will start seeing significant results within 10 weeks and greatest results by 3½ months.

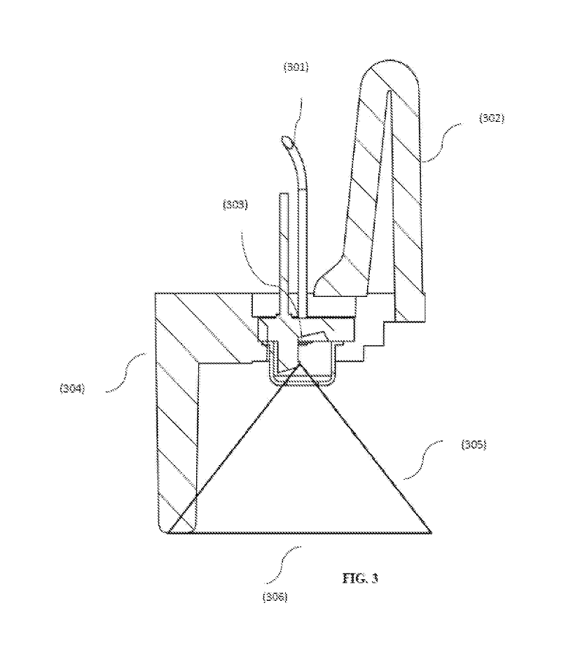

FIG. 3 illustrates the connection of Laser or LED diode or VCSEL devices and cables to shell and platform according to another embodiment of the present invention. Each Laser or LED or VCSEL (303) is connected in the center of the shell. Laser or LED diode or VCSELs shell has a spacer (304) and a small lever (302), laser or LED diode (303) and connection terminal point (302). The size of spacer (304) is keeps 1 cm for the effective treatment of scalp. Spacer (304) maintains the even distance between the laser or LED or VCSEL and scalp. Terminal point (301) is used to connect a shell with the others shell module and the power supply. Each shell has a lever (302), which is used to connect module to helmet platform. Such 25-300 tiny lasers or 25-300 LED or 25-300 VCSEL modules are fitted in the helmet. When these laser or LED or VCSEL are turns on, it produces light in the region (305) which covers 1.76 cm² areas (306) of scalp. Each module is connected in a configuration so that it is swappable with the modules of other zone.

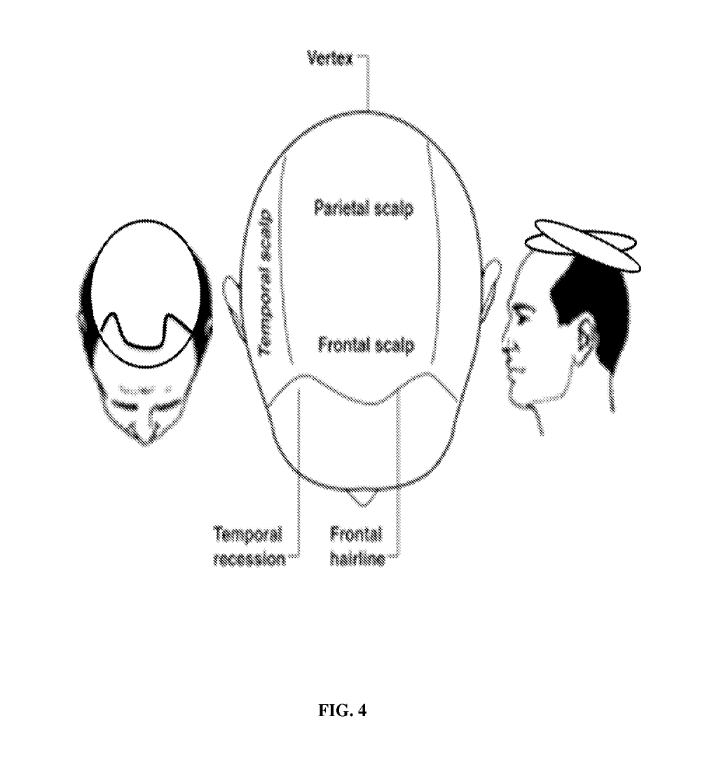

FIG. 4 illustrates a top view of human scalp according to another embodiment of present invention, which reflects different part of scalp such as front scalp, temporal scalp and frontal scalp zone, each parts represent a particular zone of scalp.

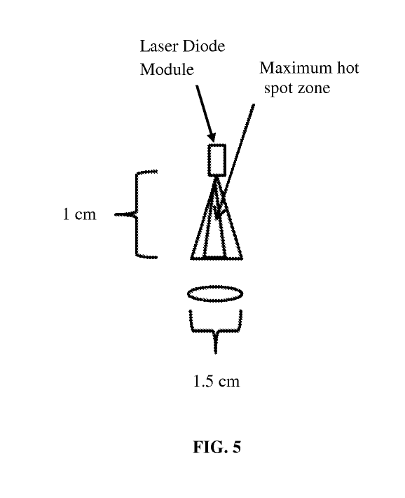

FIG. 5 illustrates a laser diode diffusion pattern according to another embodiment of present invention. In the present invention Laser or LED diode module produces light in 1.76 cm² area of scalp and distance of module is maintained by a 1 cm spacer for the effective treatment of scalp.

The helmet aids to normalize the scalp conditions mostly (dandruff, seborrhea dermatitis, Itch etc.). Another embodiment of the present invention is to provide the flexible Helmet device which helps in the decrease shedding of hair and allow hairs to grow faster, thicker and stronger (tensile strength) with an added advantage of reverse miniaturization.

The present invention provides re-growth of dormant follicles and is proven out to be more manageable. The new zone-based laser or LED or VCSEL stimulation could also be effective for tightening the surface skin of the scalp, temple and face area for other cosmetic applications because of more production of Nitric Oxide, Superoxide Dismutase, as well as increased ATP production in each of the scalp cells.

The flexible Helmet has other benefits of convenience factor (i.e. Medical facility visits are not needed any more) and can be used privately. The device is safest during usage as most laser or LED restoration devices can be harmful, if used carelessly.

The apparatus can be used in any of the comfortable places like home, bed, couch, bathroom etc., may be in a car or during work, clinic etc. The flexible Helmet device fits any head size and is adjustable. It covers the top of head or crown area (up to Norwood 5A). In a further embodiment of the present invention, the user wears the flexible Helmet 3 times a week for a minimum of 20 minutes per session (adjustable).

The present invention provides a cost effective solution for Male Pattern Baldness or Female Pattern Baldness.

Although the disclosure of apparatus has been described in connection with the embodiment of the present invention illustrated in the accompanying drawings, it is not limited thereto. It will be apparent to those skilled in the art that various substitutions, modifications and changes may be made thereto without departing from the scope and spirit of the disclosure.

What is claimed is:

1. An apparatus for therapeutic light treatment of zones of a user's scalp comprises:
at least one swappable zones to cover one or more specific zone to increase the amount of energy and for simultaneously stimulating that zone of the users scalp;
a plurality of Light emitting devices arranged inside the swappable zone;
a sensing unit configured for sensing one of a proximity of a swappable zone and generation of heat; and
a control mechanism operatively coupled to said plurality of Light emitting devices and said sensing unit for controlling the operation and providing treatment to swappable zones.

2. The apparatus of claim 1, wherein said light emitting devices are Light Emitting Diodes, Laser Diodes, or vertical-cavity surface-emitting laser (VCSEL) devices.

3. An apparatus of claim 1, wherein said sensing unit has at least one proximity sensor for sensing the swappable/treatment zone and turn on the lighting emitting devices.

4. An apparatus of claim 1, wherein said sensing unit has at least one heat sensor for regulating the temperature.

5. An apparatus of claim 1, wherein said control mechanism has at least one display unit for displaying time such as timer per session and lighting emitting devices treatment time.

6. An apparatus of claim 1, wherein said swappable zones covers specific area of scalp zone for treatment.

7. The apparatus of claim 1, further comprising a wireless control system allowing said user to turn the apparatus on and off, and to select which part of the scalp will be treated, and wherein said apparatus is powered by a battery.

8. A apparatus for therapeutic light treatment of a user's scalp, comprising:
a helmet designed to cover a user's frontal scalp, parietal scalp, and temporal scalp;
said helmet comprising a plurality of light emitting devices configured to maintain a substantially even distance from the user's scalp, and configured to expose at least said user's frontal scalp, parietal scalp, and temporal scalp to therapeutic levels of light illumination;
said helmet further comprising at least one proximity sensor to prevent operation of said plurality of light emitting devices when said helmet is not being worn; and
wherein upon placement on a user's scalp and initiation of therapeutic light treatment, at least some of said light emitting devices will operate, thus exposing at least selected regions of said user's scalp to therapeutic levels of light illumination.

9. The apparatus of claim 7, wherein said light emitting devices are Light Emitting Diodes, Laser Diodes, or vertical-cavity surface-emitting laser (VCSEL) devices.

10. The apparatus of claim 7, further comprising control mechanisms to allow at least illumination of the user's frontal scalp, parietal scalp, and temporal scalp to be controlled independently.

11. The apparatus of claim 7, further comprising at least one heat sensor to detect the temperature of the user's scalp, and a regulating mechanism designed to reduce or terminate light exposure to at least those regions of the scalp where the detected temperature exceeds a preset threshold.

12. The apparatus of claim 7, wherein said plurality of light emitting devices comprises between 25 to 300 light emitting devices.

13. The apparatus of claim 7, wherein said light emitting devices are capable of delivering between 3 to 6 joules of light energy per square centimeter of scalp during the course of a treatment session that lasts no longer than an hour.

14. The apparatus of claim 7, wherein said apparatus further comprises a computerized light emitting device control mechanism programmed to control said plurality of light emitting devices to deliver light energy in one or more patterns intended to treat male or female pattern baldness.

15. The apparatus of claim 7, wherein said apparatus covers substantially all of said user's scalp.

16. The apparatus of claim 7, wherein said apparatus covers the top of the user's head and crown area up to at least the user's Norwood 4A region.

17. The apparatus of claim 7, wherein said therapeutic light treatment is intended to be for treatment for baldness or pattern baldness.

18. The apparatus of claim 7, wherein said apparatus further comprises a display system that shows time per session and total light emitting device treatment time, and wherein said apparatus further comprises a user interface to allow said user to select said apparatus On/Off status and which part(s) of the scalp will be treated.

19. A battery powered apparatus for therapeutic light treatment of a user's scalp for baldness or pattern baldness, comprising:
a helmet designed to cover a user's frontal scalp, parietal scalp, and temporal scalp;
said helmet comprising a plurality of light emitting devices configured to maintain a substantially even distance from the user's scalp, and configured to expose at least said user's frontal scalp, parietal scalp, and temporal scalp to therapeutic levels of light illumination;
wherein said light emitting devices are between 25 to 300 Light Emitting Diodes, Laser Diodes, or vertical-cavity surface-emitting laser (VCSEL) devices capable of delivering between 3 to 6 joules of light energy per square centimeter of scalp during the course of a treatment session that lasts no longer than an hour;
said helmet further comprising at least one proximity sensor to prevent operation of said plurality of light emitting devices when said helmet is not being worn;
said helmet further comprising control mechanisms to allow at least illumination of the user's frontal scalp, parietal scalp, and temporal scalp to be controlled independently;
said helmet further comprising at least one heat sensor to detect the temperature of the user's scalp, and a regulating mechanism designed to reduce or terminate light exposure to at least those regions of the scalp where the detected temperature exceeds a preset threshold;
wherein said apparatus further comprises a display system that shows time per session and total light emitting device treatment time;
wherein said apparatus further comprises a user interface to allow said user to select apparatus On/Off status and which part(s) of the scalp will be treated; and
wherein upon placement on a user's scalp and initiation of therapeutic light treatment, at least some of said light emitting devices will operate, thus exposing at least selected regions of said user's scalp to therapeutic levels of light illumination.

20. The apparatus of claim 19, wherein said apparatus further comprises a computerized light emitting device control mechanism programmed to control said plurality of light emitting devices to deliver light energy in one or more patterns intended to treat male or female pattern baldness.
